(19) United States
(12) Patent Application Publication
(10) Pub. No.: US 2021/0000719 A1
(43) Pub. Date: Jan. 7, 2021

(54) NON-IONIC DEEP EUTECTIC MIXTURES FOR USE AS SOLVENTS AND DISPERSANTS

(71) Applicant: KALMARSUND STRATEGIC CONSULTANCY AB, Kalmar (SE)

(72) Inventors: Ian Alan NICHOLLS, Kalmar (SE); Subramanian SURIYANARAYANAN, Kalmar (SE)

(21) Appl. No.: 16/969,540

(22) PCT Filed: Feb. 22, 2019
(86) PCT No.: PCT/SE2019/050161

§ 371 Date: Aug. 12, 2020

(30) Foreign Application Priority Data
Feb. 22, 2018 (SE) 1850195-7

(51) Int. Cl. A61K 8/42; A61K 47/18; C08F 220/06; C08F 220/28; C08G 81/02; B01J 31/02; C09K 5/06; B01D 11/02

(52) U.S. Cl. CPC A61K 8/42; A61K 47/18; C08F 220/06; C08F 220/281; B01J 2231/44; B01J 31/0249; C09K 5/063; B01D 11/0288; C08G 81/028

(57) ABSTRACT

Use of a non-ionic deep eutectic mixture consisting of A and B, A being R1R2N—CO—NR3R4 and B being selected from the group consisting of R5R6N—CO—CH3 and R7R8N—CO—NR9R10, and wherein each of R1-R10 is independently H, CH3 or alkyl, as a solvent or dispersant in chemical synthesis, material synthesis or fabrication, chemical or enzymatic catalysis, food, cosmetic or pharmaceutical formulation, separation or partitioning, heat transfer, and as detergents or cleaners, as well as such mixtures, is disclosed.

### FIELD OF THE INVENTION

The present invention relates to mixtures of particular solid substances that together form non-ionic deep eutectic mixtures, which are in the liquid state at temperatures below that of the lowest-melting component, and the use of these mixtures as solvents or dispersants in applications including but not limited to: chemical synthesis, polymer synthesis, material synthesis or fabrication, chemical or enzymatic catalysis, formulation of foods, cosmetics or pharmaceuticals, separation or partitioning, heat transfer, and as detergents or cleaners.

### BACKGROUND OF THE INVENTION

Today, liquids are used extensively as solvents and dispersants in a wide variety of processes, including but not limited to: chemical synthesis, polymer synthesis, material synthesis or fabrication, chemical or enzymatic catalysis, formulation of foods, cosmetics or pharmaceutical, separation or partitioning, heat transfer, and as detergents or cleaners. The physicochemical properties of a liquid, or a liquid mixture, govern the solvent or dispersant properties of the mixture, which in turn defines their operational range in a given application. Physicochemical properties such as polarity, dielectricity and hydrogen-bonding, heat capacities and ionization capacities, etc. are inherent to a given liquid or liquid mixture, as are its toxicities.

The significance of liquid solvents and dispersants for processes important to society has driven the search for new liquids with solvation or dispersant properties better suited to particular applications, some examples include supercritical carbon dioxide and ionic liquids.

Ionic liquids are salts that are liquid at <100° C. For decades the properties of ionic liquids have been extensively explored in areas as diverse as chemical and material synthesis and drug delivery. The unique molecular-level environments for reactions provided by ionic liquids have been shown to exhibit excellent results in a range of synthesis applications.

Ionic liquids are formed from the mixing of a salt with another salt or with a substance that can act as a hydrogen bond donor, to produce a liquid with a melting point that is both less than 100° C. and below that of the constituent salt or salts. Such a mixture is described as a deep eutectic solvent (DES). Eutectic mixtures form the basis for macrostructures often encountered in surface and colloid chemistry and biology such as globular, lamellar, or rod-like structures. A number of significant drawbacks are however commonly associated with the use of ionic liquids and limit their general utility. These include their high cost of production, their high levels of toxicity, poor biodegradability and, for some applications, their high conductivities. Alternatives to ionic liquids that are devoid of these problems are therefore desirable. Accordingly, it is an object of the present invention to provide alternatives to ionic liquids which to a lesser degree suffers from at least one of these drawbacks.

It is a further object of the present invention to provide uses for such alternatives.

### SUMMARY OF THE INVENTION

At least one of the above objects, or at least one of the objects which will be evident from the below description, is according to a first aspect of the invention achieved by the use of a non-ionic deep eutectic mixture consisting of A and B, A being R¹R²N—CO—NR³R⁴ and B being selected from the group consisting of R⁵R⁶N—CO—CH₃ and R⁷R⁸N—CO—NR⁹R¹⁰, and wherein each of R¹-R¹⁰ is independently H, CH3 or alkyl, as a solvent or dispersant in chemical synthesis, material synthesis or fabrication, chemical or enzymatic catalysis, food, cosmetic or pharmaceutical formulation, separation or partitioning, heat transfer, and as detergents or cleaners.

Accordingly the present invention is based on the present inventors' further studies of a deep eutectic liquid formed upon mixing urea and acetamide in certain proportions [melting point 133° C. and 80° C. respectively, eutectic (33% urea−67% acetamide) melting point=56° C.]. Such a liquid was made in the pursuit of alternatives to imported (to the USSR) fertilizers, as described in Usanovich, M. Dok. Akad. Nauk SSSR (1958) 120, 1304-1306, however without it's properties or applications being described at that time. In addition to finding that such mixtures had uses as non-ionic deep eutectic solvents, the present inventors further, despite the difficulties in predicting deviations from normal physico-chemical properties for mixtures of unknown substances, developed a group of non-ionic deep eutectic mixtures, which, due to the low toxicities of urea and acetamide, from which the mixtures are derived, provide alternatives to traditional ionic liquids and other environmentally or economically problematic (toxic, flammable, expensive, volatile) organic solvents.

At least one of the above objects, or at least one of the objects which will be evident from the below description, is according to a second aspect of the present invention further achieved by a non-ionic deep eutectic mixture consisting of A and B, A being R¹R²N—CO—NR³R⁴ and B being selected from the group consisting of R⁵R⁶N—CO—CH₃ and R⁷R⁸N—CO—NR⁹R¹⁰, and wherein each of R¹-R¹⁰ is independently H, CH3 or alkyl, with the provisio that the non-ionic deep eutectic mixture does not comprise a mixture of urea and acetamide. As will be further described below in the detailed description and the examples, these mixtures can be used as solvents or dispersants in chemical synthesis, material synthesis or fabrication, chemical or enzymatic catalysis, food, cosmetic or pharmaceutical formulation, separation or partitioning, heat transfer, and as detergents or cleaners.

The ratio 1:2 of urea:acetamide refers to the molar ratio, i.e. 1 mole urea to 2 moles of acetamide. The molar masses of the urea and acetamide are similar, hence this ratio may alternatively be expressed as a ratio by weight.

### BRIEF DESCRIPTION OF THE DRAWINGS

A more complete understanding of the abovementioned and other features and advantages of the present invention will be apparent from the following detailed description of preferred embodiments in conjunction with the appended drawings, wherein:

FIG. 1 shows surface topography mapped using scanning electron microscopy (SEM) for the MIP film coated on Au/quartz electrosynthesized in binary eutectic solvent, and

FIG. 2 shows variation in the resonant frequency of the Au-coated quartz resonator coated with biotin imprinted polymer film prepared in binary eutectic solvent upon injection of the biotin methyl ester under flow injection analysis conditions.

### DETAILED DESCRIPTION

As the mechanism underlying the urea-acetamide deep eutectic solvent had not previously been elucidated, the present inventors used a combination of molecular modeling studies of the behavior of urea-acetamide mixtures 35:65 at over 343 K over 30 ns and statistical analyses−radial distribution studies and assessments of life times of hydrogen bonds present over the time frame. The results of these studies are presented in table 1 below:

TABLE 1
Sum of all averaged hydrogen bond occupancies
for non-ionic eutectic mixture components

| | AAM^{§} | URA^{§} |
|---|---|---|
| AAM | 40,309 | 65,832 |
| URA | | 50,939 |

# Values presented were calculated through summation of all hydrogen bond occupancies presented in the simulated system.
AAM = acetamide,
URA = urea

Statistical analysis of the molecular dynamics simulation data revealed that at the relative stoichiometry, 1:2 urea:acetamide, corresponding to that at the eutectic point, the interactions between urea and acetamide were more frequent than those between molecules of the same type. This unique insight allowed the identification of a mechanism to increase the favorability of these complexes relative to interactions between complexes, in particular selectively limiting the number hydrogen bonding sites in the participating species. This led to the design of other systems where acetamide, or acetamide derivatives, combined with urea, or urea derivatives, and urea (or derivatives) combined with urea derivatives could be predicted to have non-ionic deep eutectic behavior, see Table 2 for examples.

TABLE 2
Non-ionic deep eutectic mixtures comprised of components
A and B where the general structures of A is R¹R²N—CO—NR³R⁴ and
that of B is either R⁵R⁶N—CO—CH₃ or R⁷R⁸N—CO—NR⁹R¹⁰

| | | | | | | | | | | | mp | |
|---|---|---|---|---|---|---|---|---|---|---|---|---|
| Example | R¹ | R² | R³ | R⁴ | R⁵ | R⁶ | R⁷ | R⁸ | R⁹ | R¹⁰ | ° C. | A:B ± 3 |
| i | H | H | H | H | H | H | — | — | — | — | 56 ± 2 | 35:65 |
| ii | H | H | H | H | — | — | CH₃ | H | H | H | 61 ± 2 | 30:70 |
| iii | H | H | H | H | — | — | CH₃ | H | CH₃ | H | 69 ± 3 | 30:70 |
| iv | H | H | H | H | — | — | CH₃ | CH₃ | H | H | 97 ± 2 | 70:30 |
| v | CH₃ | H | H | H | H | H | — | — | — | — | 42 ± 3 | 50:50 |
| vi | CH₃ | H | H | H | CH₃ | H | — | — | — | — | 14 ± 2 | 80:20 |
| vii | CH₃ | H | H | H | — | — | CH₃ | CH₃ | H | H | 76 ± 4 | 80:20 |
| viii | CH₃ | H | H | H | — | — | CH₃ | H | CH₃ | H | 49 ± 3 | 50:50 |
| ix | CH₃ | CH₃ | H | H | H | H | — | — | — | — | 68 ± 3 | 80:20 |
| x | CH₃ | CH₃ | H | H | — | — | CH₃ | H | CH₃ | H | 84 ± 4 | 80:20 |
| xi | CH₃ | H | CH₃ | H | H | H | — | — | — | — | 43 ± 3 | 50:50 |
| xii | CH₃ | H | CH₃ | H | CH₃ | H | — | — | — | — | 12 ± 4 | 70:30 |

Studies of the phase behaviour of this range of systems confirmed the discovery.

Accordingly a group of non-ionic deep eutectic mixtures comprising a mixture of A and B, where A is R¹R²N—CO—NR³R⁴ and that of B is either R⁵R⁶N—CO—CH₃ or R⁷R⁸N—CO—NR⁹R¹⁰, and where each of R¹-R¹⁰ is H, CH3 or alkyl, has been found. As will be seen in the examples section further below these mixtures can be used instead of other known solvents in various applications with advantageous effects.

Thus, the first aspect of the present invention concerns use of a non-ionic deep eutectic mixture consisting of A and B, A being R¹R²N—CO—NR³R⁴ and B being selected from the group consisting of R⁵R⁶N—CO—CH₃ and R⁷R⁸N—CO—NR⁹R¹⁰, and wherein each of R¹-R¹⁰ is independently H, CH3 or alkyl, as a solvent or dispersant in chemical synthesis, material synthesis or fabrication, chemical or enzymatic catalysis, food, cosmetic or pharmaceutical formulation, separation or partitioning, heat transfer, and as detergents or cleaners.

Correspondingly, the second aspect of the present invention concerns a non-ionic deep eutectic mixture consisting of A and B, A being R¹R²N—CO—NR³R⁴ and B being selected from the group consisting of R⁵R⁶N—CO—CH₃ and R⁷R⁸N—CO—NR⁹R¹⁰, and wherein each of R¹-R¹⁰ is independently H, CH3 or alkyl, with the provisio that the non-ionic deep eutectic mixture does not comprise a mixture of urea and acetamide.

In certain embodiments of the use according to the first aspect of the present invention the non-ionic deep eutectic mixture is used as a solvent or dispersant in chemical synthesis, material synthesis or fabrication, or chemical or enzymatic catalysis.

For these applications the mixture may be N-methyl acetamide:N-methyl urea, 80:20, urea:acetamide, 1:2, or N-methyl urea:N,N′-dimethyl urea, 1:1, the ratios being molar ratios or by weight, the ratios preferably being molar ratios.

A mixture comprising N-Methyl urea (NU) and N-Methyl Acetamide (NUA) may for example be used for both solution and solid phase peptide synthesis instead of the conventional solvents N,N-dimethylformamide or dichloromethane.

In certain embodiments of the use according to the first aspect of the present invention the non-ionic deep eutectic mixture is used as a solvent or dispersant in food, cosmetic or pharmaceutical formulation.

In certain embodiments of the use according to the first aspect of the present invention the non-ionic deep eutectic mixture is used as a solvent or dispersant in separation or partitioning.

For these applications the mixture may be urea:acetamide 1:2 or N-methylurea:N-methylacetamide, 20:80, the ratios being molar ratios or by weight, the ratios preferably being molar ratios.

In certain embodiments of the use according to the first aspect of the present invention the non-ionic deep eutectic mixture is used as a solvent or dispersant in heat transfer i.e. as a heat transfer medium.

For these applications the mixture may be urea:acetamide, 1:2, the ratio being molar ratio or by weight, the ratio preferably being molar ratio.

In certain embodiments of the use according to the first aspect of the present invention the non-ionic deep eutectic mixture is used as a solvent or dispersant in detergents or cleaners.

In preferred embodiments of the non-ionic deep eutectic mixture according to the second aspect of the present invention the mixture does not comprise a 1:2 (molar ratio) mixture of urea and acetamide, preferably the mixture does not comprise a mixture of urea and acetamide, more preferably the mixture does not contain urea or acetamide, even more preferably the mixture does not comprise urea and acetamide.

Here a 1:2 mixture of urea and acetamide is to be understood to also encompass a mixture of 33 mole percent urea−67 mole percent acetamide.

In the context of the present invention the ratios and percentages given are molar ratios and mole percent if not otherwise specified. However, as the molecular masses of the components of the mixtures are similar, the ratios and percentages may alternatively be by weight.

Thus the mixture preferably does not comprise a 1:2 (by weight) mixture of urea and acetamide.

In the context of the present invention “alkyl” as a group or part of a group means a straight chain or, where available, a branched chain alkyl moiety. For example, it may represent a C1-4 alkyl.

In preferred embodiments of the use and non-ionic deep eutectic mixture according to the first and second aspects of the present invention B is R⁵R⁶N—CO—CH₃, wherein R and R⁵ are CH₃ or alkyl, R², R⁴, and R⁶ are H, and R³ is H or CH₃ or alkyl.

This provides generally lower melting points allowing the mixtures to be used in reactions or applications requiring lower temperatures.

In alternative embodiments of the use and non-ionic deep eutectic mixture according to the first and second aspects of the present invention B is R⁵R⁶N—CO—CH₃, wherein R and R⁵ are CH₃ or alkyl, R², R⁴, and R⁶ are H, and R³ is H or CH₃ or alkyl.

This provides mixtures with generally higher melting points, which may be useful for applications or reactions requiring higher temperatures.

In preferred embodiments of the use and non-ionic deep eutectic mixture according to the first and second aspects of the present invention the mixture contains 30-80% by weight of A and 70-20% by weight of B. The sum of the percentages of A and B should be 100%. In other words the percentages by weight are percentages of the total weight of A and B in the mixture.

More preferably the mixture may in some embodiments comprise 70-80% by weight of A (and thus 30-20% by weight of B). In other embodiments the mixture comprises 30-70% by weight of A (and thus 70 to 30% by weight of B).

As A and B have similar molar masses the ratio between them may alternatively be expressed by mole percent.

Thus the mixture may contain 30-80 mole percent of A and 70-20 mole percent of B. As above the sum of the percentages of A and B should be 100%. In other words the mole percentages are percentages of the total amount of moles of A and B in the mixture.

More preferably the mixture may in some embodiments comprise 70-80 mole percent of A (and thus 30-20 mole percent of B). In other embodiments the mixture comprises 30-70 mole percent of A (and thus 70-30 mole percent of B).

Preferably the mixture consists of A and B.

In certain embodiments of the use and non-ionic deep eutectic mixture according to the first and second aspects of the present invention the melting point of the mixture is 8-99° C., such as 8-71° C., such as 12-46° C.

In preferred embodiments of the use and non-ionic deep eutectic mixture according to the first and second aspects of the present invention R is CH₃, R² and R⁴ is H and R³ is H or CH₃. This provides mixtures with lower melting points. In these embodiments B is preferably R⁵R⁶N—CO—CH₃, R⁶ is H, and R⁵ is CH₃ or H, preferably CH₃. This also provides mixtures with lower melting points, especially if R⁵ is CH₃. In these embodiments the mixture preferably contains 70-80% by weight of A and 30-20% by weight of B. Alternatively in these embodiments B is R⁷R⁸N—CO—NR⁹R¹⁰, R⁷ and R⁹ is CH₃, and preferably R⁸ and R¹⁰ is H.

In certain embodiments of the use according to the first aspect of the present invention the non-ionic deep eutectic mixture comprises, contains, or consists of, urea and acetamide. Preferably the mixture comprises or contains 20-40 mole percent (or % by weight) of urea and 80-60 mole percent (or % by weight) of acetamide. More preferably the mixture comprises or contains a 1:2 (molar ratio, corresponding to 33 mole percent urea and 67 mole percent acetamide) mixture of urea and acetamide.

Following is a series of studies demonstrating the utility of these non-ionic deep eutectic mixtures as solvents or dispersants in various applications.

### Examples

### A. As Alternative to Conventional Solvents in Polymer Synthesis

Example 1: Cross-linked polymer monoliths are synthesized in the non-ionic eutectic mixture (N-methyl acetamide:N-methyl urea, 80:20 ratio by weight) described using functional monomers such as methacrylic acid (MAA) or hydroxyethylmethacrylate (HEMA) together with a cross-linking monomers, e.g. ethylene glycol dimethylacrylate (EGDMA), divinylbenzene and 1,4-bis(acryloyl)piperazine (BAP). Polymers were synthesized under thermally initiated conditions with 2,2′-azobis(2-methylpropionitrile) (AIBN) as initiator. These polymers with same Functional Monomers (EMs) and Crosslinking monomers (CLs) were also prepared in conventional solvents, in this case water, acetonitrile and toluene, to serve as control. Effects of composition of the non-ionic deep eutectic mixture in the polymerization medium on the polymer textures and structures of the synthesized polymer materials was analyzed with Brunaeur-Emmett-Teller (BET) adsorption isotherm, scanning electron microscopy (SEM), infrared spectroscopy (FTIR), surface charge and particle size and swelling rate measurements. Polymerisation was successful in both the conventional solvent and the non-ionic deep eutectic mixture, giving the same yield of polymer monolith. The materials thus prepared varied in terms of surface area, pore volume and pore diameter; 127-534 m²/g, 0.2-1.5 cm³/g and 5.2-12.6 nm, respectively. The recovery of the non-ionic deep eutectic mixture after polymerization by first extensive washing, then evaporation of the water highlighted the utility of the non-ionic deep eutectic mixture for replacing ionic liquids as well as volatile and toxic organic solvents in polymer synthesis.

### B. In Biotin-Selective (Molecularly Imprinted) Polymer Thin Film Preparation.

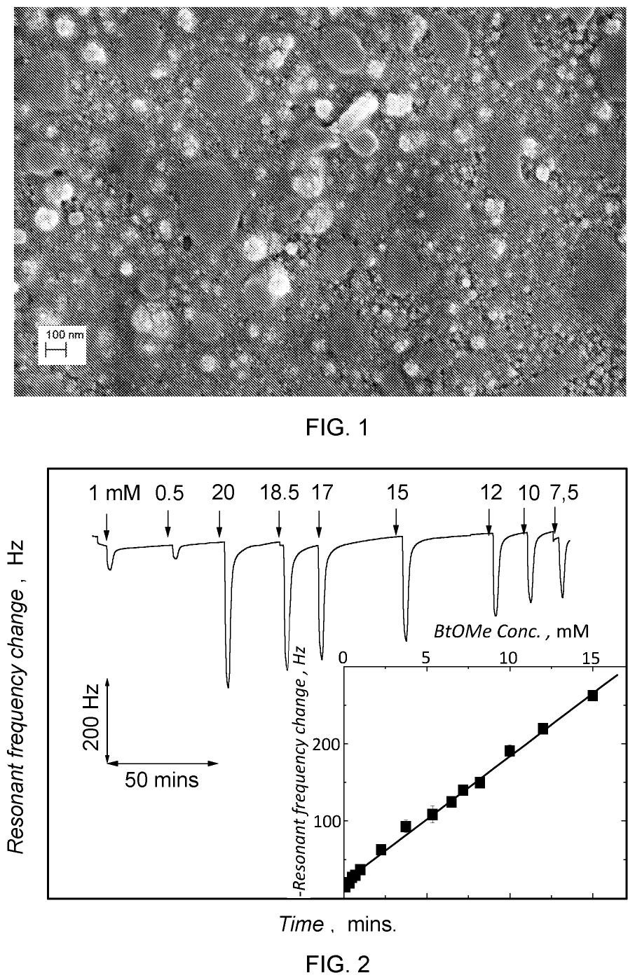

Example 2: An acetamide-urea-based non-ionic deep eutectic mixture was used in the electrochemical synthesis of thin polymer recognition films. In a typical example, cyclic voltammetric conditions were employed for the synthesis of polymer film by electrochemical co-polymerization of 16 mM of p-aminobenzoic acid (4-ABA) and 100 mM of pyrrole in the presence and absence of 4 mM biotin, in the non-ionic deep eutectic mixture of acetamide:urea in the proportions 67:33 ratio by weight on an Au/quartz electrode. Potential scan rate was 0.05 V/s and 34.6% of NH₄NO₃ was used as supporting electrolyte. Molecular imprinting of biotin (biotin being the template) using 4-ABA-pyrrole produced copolymer films displaying porous morphology, see FIG. 1 which shows surface topography of the film mapped using scanning electron microscopy (SEM) for the MIP film coated on Au/quartz electrosynthesized in binary eutectic solvent. The films synthesized with the mixture had enhanced recognition for biotin relative to those electrosynthesized using water or methanol as solvent (REF), see Table 3 below.

TABLE 3
Sensitivity and stability constants, Kₛ of the
biotin-MIP and biotin REF film interactions

| | | Correlation | |
|---|---|---|---|
| | | coefficient | |
| Recognition | Sensitivity | of | Kₛ (±st.d.) |
| film | Hz/mM | sensitivity | M⁻¹ |
| MIP film | 6.47 ± 0.56 | 0.990 | 107 |
| prepared in | | | |
| aqueous medium | | | |
| Ref film | 3.01 ± 0.32 | 0.996 | 75 |
| prepared in | | | |
| aqueous medium | | | |
| MIP film | 16.57 ± 0.27 | 0.997 | 1430 |
| prepared in | | | |
| Binary eutectic | | | |
| solvent | | | |
| Ref film | 6.68 ± 0.56 | 0.993 | 84 |
| prepared in | | | |
| Binary eutectic | | | |
| solvent | | | |

See also FIG. 2 which shows variation in the resonant frequency of the Au-coated quartz resonator coated with biotin imprinted polymer film prepared in binary eutectic solvent upon injection of the biotin methyl ester under flow injection analysis conditions.

### C. As an Alternative to Conventional Solvents in Organic Synthesis and Chemical Catalysis

Example 3: Cu-catalyzed synthesis of triazoles via the click reaction. Eutectic mixtures of N,N′-dimethylurea and N-methylurea can be employed as a medium for the Huigesan click reaction. By one-pot three-component click reaction a series of triazoles was obtained by reaction between corresponding in situ generated organic azide, and terminal alkynes.

In a typical procedure, the reaction of benzyl bromide (1), with phenyl acetylene (4) the formation of 5 was observed in the presence of catalyst, see reaction scheme below:

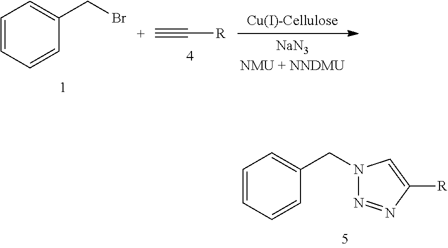

After a screening of reaction conditions in different eutectic mixtures, optimum conditions for the 1,2,3-triazole formation were identified as 1:1 w/w (i.e. ratio by weight) mixture of N-methylurea (NMU) and N,N′-dimethyl urea (NN′DMU) at 60° C. in a glass vial in presence of 5 mole % of Cu-cellulose catalyst. The reaction also proceeds in other eutectic mixtures, see table 4 below:

TABLE 4
Yields for click reaction performed using
various non-ionic eutectic mixture solvents

| | | Ratio | T | Yield | Conversion |
|---|---|---|---|---|---|
| Entry | Liquid mixture | (W:W) | (° C.) | (%) | (%) |
| 1 | Urea + | 65:35 | 80 | 80 | 90 |
| | Acetamide | | | | |
| 2 | Urea + NMU | 70:30 | 63 | 90 | 90 |
| 3 | NMU + NN′ DMU | 80:20 | 79 | 95 | 99 |
| 4 | NMU + NN′ DMU | 50:50 | 50 | 99 | 100 |
| 5 | NMA + NN′ DMU | 70:30 | 20 | 96 | 95 |

NMU = N-Methyl Urea,
NN′ DMU = N,N′ Dimethyl Urea,
NMA = N-Methyl Acetamide

In another example a mixture comprising N-Methyl urea (NMU) and N-Methyl Acetamide (NMA) is used for both solution and solid phase peptide synthesis instead of the conventional solvents N,N-dimethylformamide or dichloromethane.

### D. As an Alternative to Conventional Solvents in Extraction

### Example 4: Limonene from Lemon Peel

Finely chopped lemon peel (25 g) was added to an eutectic mixture of acetamide:urea, 67:33 (ratio by weight)(100 mL) and heated at 85° C. for 2 h. The residual lemon peel was removed by filtration. To the filtrate 300 ml (3 times the volume of eutectic mixture) of Milli-Q grade water was added and mixed vigorously to dissolve the components of the eutectic mixture. Ethyl acetate (3×20 mL) was added and the organic layer was collected separately. The organic phase was dried, filtered and evaporated to afford the limonene (200 mg) corresponding to 0.8% yield by mass. The identity of the product was confirmed by GC-MS.

### Example 5: Betulin from Birch Bark

Dry white birch bark (2.5 g) was cut and macerated and placed in a 100 ml round-bottomed flask. To that 25 ml of an eutectic mixture comprising N-methylurea:N-methylacetamide, 20:80 (ratio by weight), was added and heated at 85° C. for 2 hours. The remaining solid material was removed by filtration and the filtrate was treated with 75 ml (3 times the volume of eutectic mixture used) of Milli-Q grade water and mixed vigorously to dissolve the component of the eutectic mixture. The above solution is extracted with ethylacetate (3×20 mL) in a separating funnel and the organic layer was collected. Ethyl acetate in the organic extract was dried then removed under reduced pressure and the sample dried under vacuum before characterization by MALDI-MS and ¹H-NMR. Betulin was obtained in 400 mg (16% yield by mass).

### E. As an Alternative to Heat Transfer Agents

Example 6: A sample of an urea:acetamide, 33:67 (ratio by weight), non-ionic eutectic mixture was heated to 150° C. and the sample maintained at this temperature for 5 min before cooling until solidification. This cycle was repeated 10 times with no apparent change in the melting point of the non-ionic eutectic mixture.

1. A method comprising using a non-ionic deep eutectic mixture consisting of A and B, A being R¹R²N—CO—NR³R⁴ and B being selected from the group consisting of R⁵R⁶N—CO—CH₃ and R⁷R⁸N—CO—NR⁹R¹⁰, and wherein each of R¹-R¹⁰ is independently H, CH3 or alkyl, as a solvent or dispersant in chemical synthesis, material synthesis or fabrication, chemical or enzymatic catalysis, food, cosmetic or pharmaceutical formulation, separation or partitioning, heat transfer, and as detergents or cleaners.

2. The method according to claim 1, wherein B is R⁵R⁶N—CO—CH₃, wherein R¹ and R⁵ are CH₃ or alkyl, R², R⁴, and R⁶ are H, and R³ is H or CH₃ or alkyl.

3. The method according to claim 1, wherein B is R⁷R⁸N—CO—NR⁹R¹⁰, wherein R¹ and R² is H or CH₃ or alkyl, R₃ and R₄ is H, R⁷ is CH₃ or alkyl, R¹⁰ is H, and R⁸ and R⁹ is H or CH₃ or alkyl.

4. The method according to claim 1, wherein the mixture contains 30-80% by weight of A and 70-20% by weight of B.

5. The method according to claim 1, wherein the melting point of the mixture is 8-99° C., such as 8-71° C., such as 12-46° C.

6. The method according to claim 1, wherein R¹ is CH₃, R² and R⁴ is H and R³ is H or CH₃.

7. The method according to claim 6, wherein B is R⁵R⁶N—CO—CH₃, R⁶ is H, and R⁵ is CH₃ or H, preferably CH₃.

8. The method according to claim 7, wherein the mixture contains 70-80% by weight of A and 30-20% by weight of B.

9. The method according to claim 6, wherein B is R⁷R⁸N—CO—NR⁹R¹⁰, R⁷ and R⁹ is CH₃, and wherein preferably R⁸ and R¹⁰ is H.

10. The method according to claim 1, wherein the mixture consists of urea and acetamide.

11. A non-ionic deep eutectic mixture consisting of A and B, A being R¹R²N—CO—NR³R⁴ and B being selected from the group consisting of R⁵R⁶N—CO—CH₃ and R⁷R⁸N—CO—NR⁹R¹⁰, and wherein each of R¹-R¹⁰ is independently H, CH3 or alkyl, with the provisio that the non-ionic deep eutectic mixture does not comprise a mixture of urea and acetamide.

12. The non-ionic deep eutectic mixture according to claim 11, wherein B is R⁵R⁶N—CO—CH₃, wherein R¹ and R⁵ are CH₃ or alkyl, R², R⁴, and R⁶ are H, and R³ is H or CH₃ or alkyl.

13. The non-ionic deep eutectic mixture according to claim 11, wherein B is R⁷R⁸N—CO—NR⁹R¹⁰, wherein R¹ and R² is H or CH₃ or alkyl, R₃ and R₄ is H, R⁷ is CH₃ or alkyl, R¹⁰ is H, and R⁸ and R⁹ is H or CH₃ or alkyl.

14. The non-ionic deep eutectic mixture according to claim 11, wherein the mixture contains 30-80% by weight of A and 70-20% by weight of B, and/or wherein the melting point of the mixture is 8-99° C., such as 8-71° C., such as 12-46° C.

15. The non-ionic deep eutectic mixture according to claim 11, wherein R¹ is CH₃, R² and R⁴ is H and R³ is H or CH₃.

16. The non-ionic deep eutectic mixture according to claim 15, wherein B is R⁵R⁶N—CO—CH₃, R⁶ is H, and R⁵ is CH₃ or H, preferably CH₃.

17. The non-ionic deep eutectic mixture according to claim 16, wherein the mixture contains 70-80% by weight of A and 30-20% by weight of B.

18. The non-ionic deep eutectic mixture according to claim 15, wherein B is R⁷R⁸N—CO—NR⁹R¹⁰, R⁷ and R⁹ is CH₃, and wherein preferably R⁸ and R¹⁰ is H.
